(19) United States
(12) Patent Application Publication
(10) Pub. No.: US 2018/0215785 A1
(43) Pub. Date: Aug. 2, 2018

(54) METHOD FOR PURIFYING ANTIBODY-LIKE PROTEIN

(71) Applicant: Kaneka Corporation, Osaka (JP)

(72) Inventors: Masakatsu Nishihachijyo, Hyogo (JP); Yoshiyuki Nakano, Hyogo (JP); Fuminori Konoike, Hyogo (JP); Masayuki Takano, Hyogo (JP); Shinichi Yoshida, Hyogo (JP); Kazunobu Minakuchi, Hyogo (JP)

(73) Assignee: Kaneka Corporation, Osaka (JP)

(21) Appl. No.: 15/876,615

(22) Filed: Jan. 22, 2018

Related U.S. Application Data
(63) Continuation of application No. PCT/JP2016/071369, filed on Jul. 21, 2016.

(30) Foreign Application Priority Data
Jul. 22, 2015 (JP) 2015-145007

(51) Int. Cl. C07K 1/22; C07K 14/31

(52) U.S. Cl. CPC C07K 1/22; C07K 14/31

(57) ABSTRACT

A method for purifying an antibody-like protein includes adsorbing an antibody-like protein onto an affinity separation matrix by bringing the antibody-like protein into contact with the affinity separation matrix; and eluting the antibody-like protein by bringing an eluent having a pH of 3.5 or higher into contact with the affinity separation matrix. The affinity separation matrix includes a carrier and a ligand immobilized on the carrier, and the ligand includes an amino acid sequence derived from a sequence selected from the group consisting of SEQ ID Nos: 1 to 5. Gln or Lys in an Fc-binding site of the amino acid sequence is substituted by Ala, Ser, or Thr, and the ligand has a lower antibody-binding capacity in an acidic pH range, as compared to a ligand including the amino acid sequence without the substitution.

### TECHNICAL FIELD

One or more embodiments of the present invention relate to a method for purifying an antibody-like protein, including elution under weakly acidic conditions.

### BACKGROUND

The antibody drugs developed so far are mainly monoclonal antibodies, which are produced massively by, for example, recombinant cell culture techniques. The “monoclonal antibodies” refer to antibodies that are produced by clones of a single antibody-producing cell. Monoclonal antibodies produced by cultured cells are purified by a variety of chromatographic techniques to prepare drugs. Affinity separation chromatographic purification, particularly using immobilized Protein A, provides one-step, high-purity purification of antibodies from animal cell cultures, and thus is an essential process in the preparation of antibody drugs.

Protein A is a cell wall protein produced by the gram-positive bacterium Staphylococcus aureus, and contains a signal sequence S, five immunoglobulin-binding domains (E domain, D domain, A domain, B domain, and C domain), and a cell wall-anchoring domain known as XM region (Non-Patent Literature 1).

Many techniques have been developed for highly functionalizing Protein A by modifying it through protein engineering. Examples include techniques for improving the alkali resistance or the antibody acid dissociation properties of Protein A, and for improving its antibody-binding capacity by mutagenesis into the immobilization site (Patent Literatures 1 to 4).

The titer of antibodies produced in cell culture has been improved recently, which increases the burden on downstream purification processes. In the processes using affinity separation chromatography with immobilized Protein A, antibodies can usually be purified by binding the antibodies to the carrier at a neutral pH and then eluting the antibodies at an acidic pH. However, it is known that some antibodies form aggregates or exhibit a decrease in activity at low pH. These phenomena may not only impose burden on the purification step in antibody production (an increase in the number of steps or a decrease in yield) but also may result in serious pharmaceutical side effects. Thus, there is a need for a carrier for affinity separation chromatography that allows elution at higher pH. Known techniques for improving antibody acid dissociation properties include a substitution of Ser at position 33, a substitution of His at position 18, and substitutions of His for a variety of amino acid residues (Patent Literatures 3, 5, and 6).

Substitution mutations of Ala or Thr for Gln corresponding to position 9 of the C domain, among the Fc-binding sites of Protein A, are known, but the antibody acid dissociation properties of such variants are not disclosed (Patent Literature 7, Non-Patent Literature 2). Moreover, various variants obtained by substituting Lys corresponding to position 35 of the C domain, among the Fc-binding sites of Protein A, are also known, but the antibody acid dissociation properties of these variants are not disclosed (Patent Literature 8).

### CITATION LIST

### Patent Literature

Patent Literature 1: WO 03/080655

Patent Literature 2: EP 1123389 A

Patent Literature 3: WO 2011/118699

Patent Literature 4: WO 2012/133349

Patent Literature 5: WO 2012/087231

Patent Literature 6: WO 2012/165544

Patent Literature 7: WO 2015/005859

Patent Literature 8: JP 2007-252368 A

### Non Patent Literature

Non-Patent Literature 1: Hober S., et al., J. Chromatogr. B, 2007, vol. 848, pp. 40-47

Non-Patent Literature 2: O'Seaghdha M., et al., FEBS J, 2006, vol. 273, pp. 4831-4841

### SUMMARY

One or more embodiments of the present invention provide a method for purifying an antibody-like protein, including elution under weakly acidic conditions.

The present inventors compared and examined the activities of many recombinant Protein A variants containing amino acid substitution mutations, and found that a ligand that contains an amino acid sequence derived from any of the E, D, A, B, and C domains of Protein A of SEQ ID Nos: 1 to 5 in which Gln and/or Lys in an Fc-binding site is substituted by Ala, Ser, and/or Thr has a lower antibody-binding capacity in an acidic pH range than before the substitution.

One or more embodiments of the present invention relate to a method for purifying an antibody-like protein, including the following steps (a) and (b): (a) bringing an antibody-like protein into contact with an affinity separation matrix including a ligand immobilized on a carrier to adsorb the antibody-like protein onto the affinity separation matrix; and (b) bringing an eluent having a pH of 3.5 or higher into contact with the affinity separation matrix to elute the antibody-like protein, the ligand containing an amino acid sequence derived from any of the E, D, A, B, and C domains of Protein A of SEQ ID Nos: 1 to 5 in which Gln and/or Lys in an Fc-binding site is substituted by Ala, Ser, and/or Thr, and the ligand having a lower antibody-binding capacity in an acidic pH range than before the substitution.

The affinity separation matrix may be a carrier in which the ligand is immobilized on a water-insoluble base material.

The water-insoluble base material may be formed from a synthetic polymer or a polysaccharide.

The polysaccharide may be cellulose or agarose.

The eluent may be an acidic buffer containing at least one anion species selected from the group consisting of an acetate ion, a citrate ion, glycine, a succinate ion, a phosphate ion, and a formate ion.

The eluate may contain a reduced amount of host cell proteins and/or aggregates of the antibody-like protein of an immunoglobulin.

The elution of the antibody-like protein may be carried out by pH gradient elution.

The pH gradient elution may be carried out with an eluent having a pH of 4 to 6.

The unpurified antibody-like protein may be a mixture with host cell proteins.

The unpurified antibody-like protein may be a mixture with aggregates of the antibody-like protein.

The method for purifying an antibody-like protein according to one or more embodiments of the present invention can elute the antibody-like protein at a higher pH than in the prior art.

### BRIEF DESCRIPTION OF DRAWINGS

FIG. 1 is a table for comparison of the sequences of the E, D, A, B, and C domains of Protein A of Staphylococcus sp.

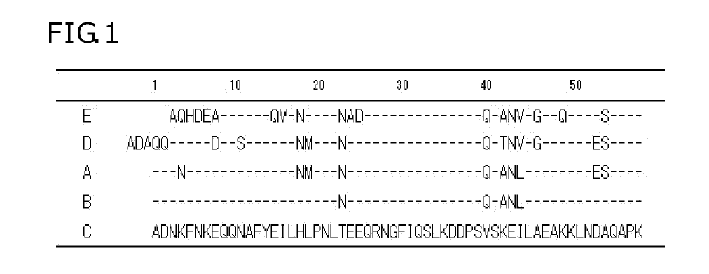

### DETAILED DESCRIPTION OF EMBODIMENTS

One or more embodiments of the present invention relate to a method for purifying an antibody-like protein, including the following steps (a) and (b): (a) bringing an antibody-like protein into contact with an affinity separation matrix including a ligand immobilized on a carrier to adsorb the antibody-like protein onto the affinity separation matrix; and (b) bringing an eluent having a pH of 3.5 or higher into contact with the affinity separation matrix to elute the antibody-like protein, the ligand containing an amino acid sequence derived from any of the E, D, A, B, and C domains of Protein A of SEQ ID Nos: 1 to 5 in which Gln and/or Lys in an Fc-binding site is substituted by Ala, Ser, and/or Thr, and the ligand having a lower antibody-binding capacity in an acidic pH range than before the substitution.

Protein A is a protein including the immunoglobulin-binding E, D, A, B, and C domains. The E, D, A, B, and C domains are immunoglobulin-binding domains capable of binding to regions other than the complementarity determining regions (CDRs) of immunoglobulins, and each domain has activity to bind to the Fc and Fab regions of immunoglobulins and particularly to the Fv regions of the Fab regions. In one or more embodiments of the present invention, the Protein A may be derived from any source, but may be derived from Staphylococcus species.

The term “protein” is intended to include any molecule having a polypeptide structure and also encompass fragmentized polypeptide chains and polypeptide chains linked by peptide bonds. The term “domain” refers to a higher-order protein structural unit having a sequence that consists of several tens to hundreds of amino acid residues, enough to fulfill a certain physicochemical or biochemical function.

The domain-derived amino acid sequence means an amino acid sequence before the amino acid substitution. The domain-derived amino acid sequence is not limited only to the wild-type amino acid sequences of the E, D, A, B, and C domains of Protein A, and may include any amino acid sequence partially engineered by amino acid substitution, insertion, deletion, or chemical modification, provided that it forms a protein having the ability to bind to an Fc region. Examples of the domain-derived amino acid sequence include the amino acid sequences of the E, D, A, B, and C domains of Staphylococcus Protein A of SEQ ID NOs: 1 to 5. Examples also include proteins having amino acid sequences obtained by introducing a substitution of Ala for Gly corresponding to position 29 of the C domain into the E, D, A, B, and C domains of Protein A. In addition, the Z domain produced by introducing A1V and G29A mutations into the B domain corresponds to the domain-derived amino acid sequence because it also has the ability to bind to an Fc region. The domain-derived amino acid sequence may be a domain having high chemical stability or a variant thereof.

The domain-derived amino acid sequence has the ability to bind to an Fc region. The domain-derived amino acid sequence may have a sequence identity of 85% or higher, 90% or higher, or 95% or higher, to any of the E, D, A, B, and C domains of Protein A of SEQ ID NOs: 1 to 5.

The ligand used in one or more embodiments of the present invention contains an amino acid sequence derived from any of the E, D, A, B, and C domains of Protein A of SEQ ID Nos: 1 to 5 in which Gln and/or Lys in an Fc-binding site is substituted by Ala, Ser, and/or Thr.

In the amino acid sequence derived from any of the E, D, A, B, and C domains of Protein A of SEQ ID Nos: 1 to 5, the Fc-binding site means the amino acid residues corresponding to positions 5, 9, 10, 11, 13, 14, 17, 28, 31, 32, and 35 of the C domain of Protein A (Proc. Natl. Acad. Sci. USA, 2000, vol. 97, pp. 5399-5404).

Examples of the Gln in the Fc-binding site include amino acid residues corresponding to positions 9, 10, and 32 of the C domain. Among them, amino acid residues corresponding to positions 9 and 32 of the C domain may be used.

Examples of the Lys in the Fc-binding site include amino acid residues corresponding to position 35 of the C domain.

The amino acid substitution means a mutation which deletes the original amino acid and adds a different type of amino acid to the same position. It should be noted that amino acid substitutions are denoted herein with the code for the wild-type or non-mutated type amino acid, followed by the position number of the substitution, followed by the code for changed amino acid. For example, a substitution of Ala for Gly at position 29 is represented by G29A.

Examples of amino acids that may substitute the Gln and/or Lys in the Fc-binding site include Ala, Ser, and Thr.

More specific substitution embodiments include substitutions of Ala for Gln corresponding to position 9, Ser for Gln corresponding to position 9, Thr for Gln corresponding to position 9, Ala or Thr for Gln corresponding to position 32, and Ser for Lys corresponding to position 35 of the C domain. Among these Q9A, Q9S, Q9T, Q32A, Q32T, and K35S in the C domain may be used.

Any number of amino acids may be substituted as long as the antibody-binding capacity in an acidic pH range is lower than before substitution. In order to maintain the conformation of the protein before mutagenesis, the number of amino acid substitutions may be 4 or less, or 2 or less.

As long as the antibody-binding capacity in an acidic pH range is lower than before substitution, the ligand may contain any amino acid substitution, in addition to the substitution of Gln and/or Lys in the Fc-binding site by Ala, Ser, and/or Thr. Examples of such amino acid substitutions include G29A, F5A, F5Y, A12R, F13Y, L17I, L17T, L17V, L19R, L22R, Q26R, I31L, I31S, I31T, I31V, Q32R, S33H, V40Q, V40T, and V40H substitutions in the C domain. Examples also include similar substitutions of amino acids corresponding to the foregoing positions of the C domain in the E, D, A, and B domains. Amino acid substitutions that substitute Asn by a different amino acid may be used because they can be expected to improve alkali resistance.

The amino acid sequence derived from any of the E, D, A, B, and C domains of Protein A of SEQ ID NOs: 1 to 5 in which Gln and/or Lys in an Fc-binding site is substituted by Ala, Ser, and/or Thr may have a sequence identity of 85% or higher, 90% or higher, or 95% or higher, to any of the E, D, A, B, and C domains of Protein A of SEQ ID NOs: 1 to 5.

The ligand used in one or more embodiments of the present invention may retain at least 90%, or at least 95%, of the following amino acid residues: Gln-9, Gln-10, Phe-13, Tyr-14, Leu-17, Pro-20, Asn-21, Leu-22, Gln-26, Arg-27, Phe-30, Ile-31, Leu-34, Pro-38, Ser-39, Leu-45, Leu-51, Asn-52, Gln-55, and Pro-57 (the residue numbers indicated are for the C domain).

The ligand used in one or more embodiments of the present invention is characterized by having a lower antibody-binding capacity in an acidic pH range than before substitution. The acidic pH range may be a weakly acidic range, specifically with a pH in the range of 3 to 6.

The antibody-binding capacity in the acidic range can be evaluated by a pH gradient elution test using IgG Sepharose (Example 1) or by measurement of the antibody-binding capacity in an acidic pH range using an intermolecular interaction analyzer. For example, in the case of a pH gradient elution test using IgG Sepharose, a variant that has a lower antibody-binding capacity in an acidic range than that of the non-mutated protein (e.g. C-G29A.2d) elutes at higher pH. When the elution pH calculated from the top of the elution peak of the non-mutated protein is taken as reference, the elution pH of the variant may be higher than the reference by 0.05 or more, or by 0.1 or more.

The ligand used in one or more embodiments of the present invention may be a ligand consisting only of a single domain in which the amino acid substitution is introduced, or a multi-domain ligand obtained by linking at least two domains in which the amino acid substitution is introduced.

In the case of a multi-domain ligand, the ligand may be a ligand consisting of the same domains (a homopolymer such as a homodimer or homotrimer) or a ligand consisting of different domains (a heteropolymer such as a heterodimer or heterotrimer). The number of domains linked may be 2 or more, 2 to 10, or 2 to 6.

In the multi-domain ligand, the monomeric domains may be linked to each other by, for example, but not limited to: a method that does not use an amino acid residue as a linker; or a method that uses one or more amino acid residues. The number of amino acid residues used for linkage is not particularly limited. The linkage mode and the number of linkages are also not particularly limited, provided that the three-dimensional conformation of the monomeric domains does not become unstable.

A fusion protein in which the ligand, as a constituent component, is fused with another protein having a different function may also be used in one or more embodiments of the present invention. Examples of the fusion protein include, but are not limited to, those fused with albumin, GST (glutathione S-transferase), or MBP (maltose-binding protein). Expression as a fusion protein with GST or MBP facilitates purification of the ligand. The ligand may also be fused with a nucleic acid such as a DNA aptamer, a drug such as an antibiotic, or a polymer such as polyethylene glycol (PEG).

The DNA encoding the ligand may be any DNA having a base sequence that is translated into the amino acid sequence of the ligand. Such a base sequence can be obtained by common known techniques, such as polymerase chain reaction (hereinafter abbreviated as PCR). Alternatively, it can be synthesized by known chemical synthesis techniques or may be available from DNA libraries. A codon in the base sequence may be replaced with a degenerate codon, and the base sequence is not necessarily the same as the original base sequence, provided that the coding base sequence is translated into the same amino acids.

The DNA in one or more embodiments of the present invention can be obtained by site-directed mutagenesis of a conventionally known DNA encoding a wild-type or mutated Protein A domain. Site-directed mutagenesis may be performed by, for example, recombinant DNA technology or PCR as follows.

In the case of mutagenesis by recombinant DNA technology, for example, if there are suitable restriction enzyme recognition sequences on both sides of a mutagenesis target site in the gene encoding the ligand, a cassette mutagenesis method can be used in which these restriction enzyme recognition sites are cleaved with the restriction enzymes to remove a region containing the mutagenesis target site, and a DNA fragment in which only the target site is mutated by chemical synthesis or other methods is then inserted.

In the case of site-directed mutagenesis by PCR, for example, a double primer method can be used in which PCR is performed using a double-stranded plasmid encoding the ligand as a template and two synthetic oligo primers containing complementary mutations in the + and − strands.

In one or more embodiments, a DNA encoding the multi-domain ligand can be prepared by ligating the desired number of DNAs encoding the monomeric ligand (single domain) in tandem. For example, the DNA encoding the multi-domain ligand may be prepared by a ligation method in which a suitable restriction enzyme site is introduced into a DNA sequence, which is then cleaved with the restriction enzyme into a double-stranded DNA fragment, followed by ligation using a DNA ligase. A single restriction enzyme site or a plurality of different restriction enzyme sites may be introduced. Alternatively, the DNA encoding the multi-domain ligand may be prepared by applying any of the mutagenesis methods to a DNA encoding Protein A (e.g., see WO 06/004067). Here, if the base sequences each encoding a monomeric ligand in the DNA encoding the multi-domain ligand are the same, then homologous recombination may be induced in host cells. For this reason, the ligated DNAs encoding a monomeric ligand may have 90% or lower, or 85% or lower base sequence identity.

The vector in one or more embodiments of the present invention includes a base sequence encoding the above-described ligand or multi-domain ligand, and a promoter that is operably linked to the base sequence to function in a host cell. Typically, the vector can be constructed by linking or inserting the above-described DNA encoding the ligand into a vector.

The vector used for insertion of the gene is not particularly limited, provided that it is capable of autonomous replication in a host cell. The vector may be a plasmid DNA or phage DNA. When Escherichia coli is used as a host cell, examples of the vector used for insertion of the gene include pQE vectors (QIAGEN), pET vectors (Merck), and pGEX vectors (GE Healthcare, Japan). When Brevibacillus is used as a host cell, examples include the known Bacillus subtilis vector pUB110 and pHY500 (JP H02-31682 A), pNY700 (JP H04-278091 A), pNU211R2L5 (JP H07-170984 A), pHT210 (JP H06-133782 A), and the shuttle vector pNCMO2 between Escherichia coli and Brevibacillus (JP 2002-238569 A).

A transformant can be produced by transforming a host cell with the vector. Any host cell may be used. For low-cost mass production, Escherichia coli, Bacillus subtilis, and bacteria (eubacteria) of genera including Brevibacillus, Staphylococcus, Streptococcus, Streptomyces, and Corynebacterium can be suitably used. Gram-positive bacteria such as Bacillus subtilis and bacteria of the genera Brevibacillus, Staphylococcus, Streptococcus, Streptomyces, and Corynebacterium may be used. Bacteria of the genus Brevibacillus, which are known for their application in mass production of Protein A (WO 06/004067), may also be used.

Examples of the bacteria of the genus Brevibacillus include, but are not limited to: Brevibacillus agri, B. borstelensis, B. brevis, B. centrosporus, B. choshinensis, B. formosus, B. invocatus, B. laterosporus, B. limnophilus, B. parabrevis, B. reuszeri, and B. thermoruber. Examples include Brevibacillus brevis 47 (JCM6285), Brevibacillus brevis 47K (FERM BP-2308), Brevibacillus brevis 47-5Q (JCM8970), Brevibacillus choshinensis HPD31 (FERM BP-1087), and Brevibacillus choshinensis HPD31-OK (FERM BP-4573). Mutants (or derivative strains) such as protease-deficient strains, high-expressing strains, or sporulation-deficient strains of the Brevibacillus bacteria may be used for purposes such as improved yield. Specific examples include the protease mutant Brevibacillus choshinensis HPD31-OK (JP H06-296485 A) and sporulation-deficient Brevibacillus choshinensis HPD31-SP3 (WO 05/045005), which are derived from Brevibacillus choshinensis HPD31.

The vector may be introduced into the host cell by, for example, but not limited to: a calcium ion method, an electroporation method, a spheroplast method, a lithium acetate method, an agrobacterium infection method, a particle gun method, or a polyethylene glycol method. Moreover, in one or more embodiments, the obtained gene function may be expressed in the host cell, for example, by incorporating the gene into a genome (chromosome).

The transformant, or a cell-free protein synthesis system including the DNA can be used to produce the ligand.

In the case where the transformant is used to produce the ligand, the transformed cell may be cultured in a medium to produce and accumulate the ligand in the cultured cells (including the periplasmic space thereof) or in the culture medium (extracellularly), and the desired ligand can be collected from the culture.

When the transformed cell is used to produce the ligand, the ligand may be accumulated within the transformant cell and/or in the periplasmic space thereof. In this case, the accumulation within the cell is advantageous in that the expressed protein can be prevented from oxidation, and there are no side reactions with the medium components. On the other hand, the accumulation in the periplasmic space is advantageous in that decomposition by intracellular proteases can be suppressed. Alternatively, the ligand may be produced by secreting the ligand extracellularly of the transformant. This does not require cell disruption and extraction steps and is thus advantageous for reducing production costs.

The transformed cell in one or more embodiments of the present invention can be cultured in a medium according to common methods for culturing host cells. The medium used for culturing the transformant is not particularly limited, provided that it allows for high yield and high efficiency production of the ligand. Specifically, carbon and nitrogen sources such as glucose, sucrose, glycerol, polypeptone, meat extracts, yeast extracts, and casamino acids can be used. In addition, the medium is supplemented with inorganic salts such as potassium salts, sodium salts, phosphates, magnesium salts, manganese salts, zinc salts, or iron salts, as necessary. In the case of an auxotrophic host cell, nutritional substances necessary for its growth may be added. Moreover, antibiotics such as penicillin, erythromycin, chloramphenicol, and neomycin may optionally be added.

Furthermore, a variety of known protease inhibitors, phenylmethane sulfonyl fluoride (PMSF), benzamidine, 4-(2-aminoethyl)-benzenesulfonyl fluoride (AEBSF), antipain, chymostatin, leupeptin, pepstatin A, phosphoramidon, aprotinin, and ethylenediaminetetraacetic acid (EDTA), and/or other commercially available protease inhibitors may be added at appropriate concentrations in order to reduce the degradation or molecular-size reduction of the ligand caused by host-derived proteases present inside or outside the cells.

In order to ensure accurate folding of the ligand, molecular chaperones such as GroEL/ES, Hsp70/DnaK, Hsp90, or Hsp104/ClpB may be used. In this case, for example, they can be allowed to coexist with the ligand by, for example, co-expression or incorporation into a fusion protein. Other methods for ensuring accurate folding of the ligand may also be used such as, but not limited to, adding an additive for assisting accurate folding to the medium or culturing at low temperatures.

Examples of media that can be used to culture the transformed cell obtained using Escherichia coli as a host include LB medium (1% triptone, 0.5% yeast extract, 1% NaCl) and 2×YT medium (1.6% triptone, 1.0% yeast extract, 0.5% NaCl).

Examples of media that can be used to culture the transformant obtained using Brevibacillus as a host include TM medium (1% peptone, 0.5% meat extract, 0.2% yeast extract, 1% glucose, pH 7.0) and 2SL medium (4% peptone, 0.5% yeast extract, 2% glucose, pH 7.2).

The cell may be aerobically cultured at a temperature of 15° C. to 42° C., or 20° C. to 37° C., for several hours to several days under aeration and stirring conditions to accumulate the ligand in the cultured cells (including the periplasmic space thereof) or in the culture medium (extracellularly), followed by recovery of the ligand. In some cases, the cell may be cultured anaerobically without air.

In the case where the recombinant protein is secreted, the produced recombinant protein can be recovered after the culture by separating the cultured cells from the supernatant containing the secreted protein by a common separation method such as centrifugation or filtration.

Also in the case where the ligand is accumulated in the cultured cells (including the periplasmic space), the ligand accumulated in the cells can be recovered, for example, by collecting the cells from the culture medium, e.g. via centrifugation or filtration, followed by disrupting the cells, e.g. via sonication or French press, and/or solubilizing the ligand with, for example, a surfactant.

In the case where the ligand is produced using a cell-free protein synthesis system, the cell-free protein synthesis system is not particularly limited. Examples include synthesis systems derived from procaryotic cells, plant cells, or higher animal cells.

The ligand can be purified by methods such as affinity chromatography, cation or anion exchange chromatography, and gel filtration chromatography, used alone or in an appropriate combination.

Whether the purified product is the target ligand may be confirmed by common techniques such as SDS polyacrylamide gel electrophoresis, N-terminal amino acid sequencing, or Western blot analysis.

An affinity separation matrix can be prepared by immobilizing the ligand used in one or more embodiments of the present invention as an affinity ligand onto a carrier made of a water-insoluble base material. The term “affinity ligand” means a substance (functional group) that selectively captures (binds to) a target molecule from a mixture of molecules by virtue of a specific affinity between the molecules such as antigen-antibody binding, and refers herein to a protein that specifically binds to an immunoglobulin. The term “ligand” as used alone herein is synonymous with “affinity ligand”.

Examples of the carrier made of a water-insoluble base material include inorganic carriers such as glass beads and silica gel; organic carriers such as synthetic polymers (e.g. cross-linked polyvinyl alcohol, cross-linked polyacrylate, cross-linked polyacrylamide, cross-linked polystyrene) and polysaccharides (e.g. cellulose, agarose, cross-linked dextran); and composite carriers formed by combining these carriers such as organic-organic or organic-inorganic composite carriers.

Examples of commercially available products include GCL2000 (porous cellulose gel), Sephacryl S-1000 (prepared by covalently cross-linking allyl dextran with methylene bisacrylamide), Toyopearl (methacrylate carrier), Sepharose CL4B (cross-linked agarose carrier), and Cellufine (cross-linked cellulose carrier), although the water-insoluble carrier used in one or more embodiments of the present invention is not limited to the carriers listed above.

In view of the purpose and method of using the affinity separation matrix, the water-insoluble carrier should have a large surface area and may be a porous material having a large number of fine pores of an appropriate size. The carrier may be in any form such as bead, monolith, fiber, film (including hollow fiber) or other optional forms.

The immobilization of the ligand onto the carrier may be carried out by, for example, conventional coupling methods utilizing an amino, carboxyl, or thiol group on the ligand. Such coupling may be accomplished by an immobilization method that includes reacting the carrier with cyanogen bromide, epichlorohydrin, diglycidyl ether, tosyl chloride, tresyl chloride, hydrazine, sodium periodate, or the like to activate the carrier (or introduce a reactive functional group into the carrier surface), and performing a coupling reaction between the carrier and the compound to be immobilized as a ligand; or an immobilization method that includes adding a condensation reagent such as carbodiimide or a reagent having a plurality of functional groups in the molecule such as glutaraldehyde to a system containing the carrier and the compound to be immobilized as a ligand, followed by condensation and cross-linking.

A spacer molecule consisting of a plurality of atoms may be introduced between the ligand and the carrier, or alternatively, the ligand may be directly immobilized onto the carrier. Accordingly, for immobilization, the ligand may be chemically modified or may incorporate an additional amino acid residue useful for immobilization. Examples of amino acids useful for immobilization include amino acids having in a side chain a functional group useful for a chemical reaction for immobilization, such as Lys which contains an amino group in a side chain, and Cys which contains a thiol group in a side chain. Any modification or alteration may be made for immobilization, as long as the effect provided to the ligand is also provided to the matrix in which the ligand is immobilized on the water-insoluble carrier.

Examples of the antibody-like protein to be purified by one or more embodiments of the present invention include, but are not limited to, immunoglobulin G and immunoglobulin G derivatives.

Examples of the immunoglobulin G include human IgG1, IgG2, and IgG4, mouse IgG1, IgG2 A, IgG2 B, and IgG3, rat IgG1 and IgG2 C, goat IgG1 and IgG2, guinea pig IgG, bovine IgG2, and rabbit IgG. Examples of the immunoglobulin G derivatives include chimeric immunoglobulin G in which the domains of human immunoglobulin G are partially replaced and fused with immunoglobulin G domains of another biological species, humanized immunoglobulin G in which complementarity determining regions (CDRs) of human immunoglobulin G are replaced and fused with antibody CDRs of another biological species, immunoglobulin G in which a sugar chain in the Fc region is molecularly altered, and artificial immunoglobulin G in which the Fv and Fc regions of human immunoglobulin G are fused.

As described earlier, the regions to which the ligand binds are broadly specified as Fab regions (particularly Fv regions) and Fc regions. However, since the conformation of antibodies is already known, the proteins to which the ligand and the affinity separation matrix bind may be ones obtained by further altering (e.g. fragmentizing) the Fab or Fc regions while maintaining the conformation of the regions to which Protein A binds by protein engineering techniques.

The antibody-like protein can be purified by the steps of: bringing the antibody-like protein into contact with an affinity separation matrix including the ligand immobilized on a carrier to adsorb the antibody-like protein onto the affinity separation matrix; and bringing an eluent having a pH of 3.5 or higher into contact with the affinity separation matrix to elute the antibody-like protein.

In the first step of the method for purifying an antibody-like protein, the antibody-like protein is brought into contact with an affinity separation matrix including the ligand immobilized on a carrier to adsorb the antibody-like protein onto the affinity separation matrix. Specifically, a buffer containing the antibody-like protein is adjusted to be neutral, and the resulting solution is passed through an affinity column filled with the affinity separation matrix to adsorb the antibody-like protein. Examples of the buffer include citric acid, 2-(N-morpholino)ethanesulfonic acid (MES), Bis-Tris, N-(2-acetamido)iminodiacetic acid (ADA), piperazine-1,4-bis(2-ethanesulfonic acid) (PIPES), N-(2-acetamido)-2-aminoethanesulfonic acid (ACES), 3-(N-morpholino)-2-hydroxypropanesulfonic acid (MOPSO), N,N-bis(2-hydroxyethyl)-2-aminoethanesulfonic acid (BES), 3-(N-morpholino)propanesulfonic acid (MOPS), N-tris(hydroxymethyl)methyl-2-aminoethanesulfonic acid (TES), 4-(2-hydroxyethyl)-1-piperazineethanesulfonic acid (HEPES), triethanolamine, 3-[4-(2-hydroxyethyl)-1-piperazinyl]propanesulfonic acid (EPPS), Tricine, Tris, glycylglycine, Bicine, N-tris(hydroxymethyl)methyl-3-aminopropanesulfonic acid (TAPS), and Dulbecco's phosphate buffered saline. The pH at which the antibody-like protein is adsorbed onto the affinity separation matrix may be 6.5 to 8.5, or 7 to 8. The temperature at which the antibody-like protein is adsorbed onto the affinity separation matrix may be 1° C. to 40° C., or 4° C. to 30° C.

The first step may be followed by passing an appropriate amount of pure buffer through the affinity column to wash the inside of the column. At this point, the desired antibody-like protein remains adsorbed on the affinity separation matrix in the column. The buffer for washing may be the same as the buffer used in the first step.

In the second step of the method for purifying an antibody-like protein, an eluent having a pH of 3.5 or higher is brought into contact with the affinity separation matrix to elute the antibody-like protein. Examples of the eluent include those containing an anion species such as an acetate ion, a citrate ion, glycine, a succinate ion, a phosphate ion, a formate ion, a propionate ion, γ-aminobutyrate, or lactate.

The pH of the eluent may be 3.5 or higher, 3.6 or higher, 3.75 or higher, 3.8 or higher, 3.9 or higher, or 4.0 or higher. The upper limit of the pH of the eluent may be 6.0.

The elution of the antibody-like protein from the affinity separation matrix may also be carried out in a stepwise manner using different pH eluents. Moreover, gradient elution with a pH gradient using two or more eluents with different pH values (e.g. pH 6 and pH 3) is suitable because higher purification can be achieved. Since the affinity separation matrix in one or more embodiments of the present invention allows elution of the antibody under particularly high pH conditions, the eluents in the gradient elution may partially include an eluent having a pH of 4 to 6. A surfactant (such as Tween 20 or Triton-X 100), a chaotropic agent (such as urea or guanidine), or an amino acid (such as arginine) may also be added to the buffer used for adsorption, washing, or elution.

Similarly, the pH in the affinity column filled with the affinity separation matrix at the time of elution of the antibody-like protein may be 3.5 or higher, 3.6 or higher, 3.75 or higher, 3.8 or higher, 3.9 or higher, or 4.0 or higher. When elution is performed at a pH of 3.5 or higher, damage to the antibody can be reduced (Ghose S. et al., Biotechnology and bioengineering, 2005, vol. 92, No. 6). The upper limit of the pH in the affinity column filled with the affinity separation matrix at the time of elution of the antibody-like protein may be 6.0. According to the purification method in one or more embodiments of the present invention, the antibody-like protein can be dissociated under acidic elution conditions closer to neutral, so that a sharper elution peak profile can be obtained when the antibody-like protein is eluted under acidic conditions. Due to the sharper chromatographic elution peak profile, a smaller volume of eluent can be used to recover an eluate having a higher antibody concentration.

The temperature when the antibody-like protein is eluted may be 1° C. to 40° C., or 4° C. to 30° C.

The percent recovery of the antibody-like protein recovered by the purification method according to one or more embodiments of the present invention may be 90% or higher, or 95% or higher. The percent recovery may be calculated using the following equation:

Percent recovery (%)=[(concentration (mg/mL) of eluted antibody-like protein)×(volume (ml) of eluted liquid)]/[(concentration (mg/mL) of loaded antibody-like protein)×(volume (ml) of loaded liquid)]×100.

According to the purification method in one or more embodiments of the present invention, it is possible to reduce contamination of host cell proteins for expressing the antibody-like protein. It is also possible to reduce contamination of aggregates of the antibody-like protein. The contamination of these proteins may increase the burden on the purification step in antibody-like protein production (an increase in the number of steps or a decrease in yield), and may also result in serious pharmaceutical side effects due to the impurity proteins. In contrast, the purification method according to one or more embodiments of the present invention using a higher pH eluent can avoid these contaminations.

Also, when the unpurified antibody-like protein is a mixture with host cell proteins, the affinity separation matrix in one or more embodiments of the present invention is effective in separating the antibody-like protein from the host cell proteins. The host cell from which the host cell proteins originate is a cell capable of expressing the antibody-like protein, such as particularly a CHO cell or Escherichia coli, for which gene recombination techniques have been established. Such host cell proteins can be quantified using commercially available immunoassay kits. For example, CHO cell proteins may be quantified with CHO HCP ELISA kit (Cygnus).

Also when the unpurified antibody-like protein is a mixture with aggregates of the antibody-like protein, the affinity separation matrix in one or more embodiments of the present invention is effective in purifying the non-aggregated antibody-like protein from a solution containing aggregates of the antibody-like protein, e.g. in an amount of at least 1%, 5%, or 10% of the total amount of the antibody-like protein in the eluate, to remove the aggregates. The amount of the aggregates may be analyzed and quantified by, for example, gel filtration chromatography.

The affinity separation matrix can be reused by passing through it a pure buffer having an appropriate strong acidity or strong alkalinity which does not completely impair the functions of the ligand compound and the carrier base material (or optionally a solution containing an appropriate modifying agent or an organic solvent) for washing.

The affinity of the ligand and the affinity separation matrix for the antibody-like protein may be tested using, for example, biosensors such as Biacore system (GE Healthcare, Japan) based on the principle of surface plasmon resonance. When the affinity of the ligand for an immunoglobulin is measured as an affinity for a human immunoglobulin G preparation using the Biacore system, which will be described later, the association constant (K_{A}) may be 10⁶ (M⁻¹) or higher, or 10⁷ (M⁻¹) or higher.

The measurement may be carried out under any conditions that allow detection of a binding signal corresponding to the binding of the ligand to the immunoglobulin Fc region. The affinity can be easily evaluated at a (constant) temperature of 20° C. to 40° C. and a neutral pH of 6 to 8.

Examples of immunoglobulin molecules that can be used as binding partners include gammaglobulin “Nichiyaku” (human immunoglobulin G, Nihon Pharmaceutical Co. Ltd.) which is a polyclonal antibody, and commercially available pharmaceutical monoclonal antibodies.

A skilled person can easily evaluate the difference in affinity by preparing and analyzing sensorgrams of binding to the same immunoglobulin molecule under the same measurement conditions, and using the obtained binding parameters to make a comparison with the control ligand.

Examples of binding parameters that can be used include association constant (K_{A}) and dissociation constant (K_{D}) (Nagata et al., “Real-time analysis of biomolecular interactions”, Springer-Verlag Tokyo, 1998, page 41). The association constant between the ligand and Fab may be determined in an experimental system using Biacore system in which an Fab fragment of an immunoglobulin of the VH3 subfamily is immobilized on a sensor chip, and the ligand is added to a flow channel at a temperature of 25° C. and a pH of 7.4. Although the association constant may also be described as affinity constant in some documents, the definitions of these terms are essentially the same.

### EXAMPLES

The following description is offered to illustrate one or more embodiments of the present invention in greater detail by reference to examples, but the scope of the present invention is not limited to these examples. In the examples, operations such as recombinant DNA production and engineering were performed in accordance with the following textbooks, unless otherwise noted: (1) T. Maniatis, E. F. Fritsch, J. Sambrook, “Molecular Cloning/A Laboratory Manual”, 2nd edition (1989), Cold Spring Harbor Laboratory (USA); (2) Masami Muramatsu, “Lab Manual for Genetic Engineering”, 3rd edition (1996), Maruzen Co., Ltd. The materials such as reagents and restriction enzymes used in the examples were commercially available products, unless otherwise specified.

Proteins obtained in the examples are represented by “an alphabetical letter identifying the domain—an introduced mutation (wild for the wild type)”. For example, the wild-type C domain of Protein A is represented by “C-wild”, and a C domain variant containing G29E mutation is represented by “C-G29E”. Variants containing two mutations together are represented by indicating both with a slash. For example, a C domain variant containing G29E and S13L mutations is represented by “C-G29E/S13L”. Proteins consisting of a plurality of single domains linked together are represented by adding a period (.) followed by the number of linked domains followed by “d”. For example, a protein consisting of five linked C domain variants containing G29E and S13L mutations is represented by “C-G29E/S13L.5d”.

### Example 1

### Evaluation of Antibody-Binding Capacity of C Domain Variant using IgG-Immobilized Carrier

The total synthesis of artificially synthesized genes of engineered C-G29A.2d variants was outsourced to Eurofins Genomics K.K. These genes were synthesized by introducing amino acid substitution mutations as shown in Table 1 into a DNA (SEQ ID NO: 7) obtained by adding PstI and XbaI recognition sites to the 5′ and 3′ ends, respectively, of a DNA encoding C-G29A.2d (SEQ ID NO: 6) containing G29A mutation in the C domain of Protein A. They were subcloned into expression plasmids, which were then digested with the restriction enzymes PstI and XbaI (Takara Bio, Inc.), and each of the obtained DNA fragments was ligated to a Brevibacillus expression vector pNCMO2 (Takara Bio, Inc.) digested with the same restriction enzymes to construct expression plasmids in which a DNA encoding the amino acid sequence of each engineered C-G29A.2d was inserted into a Brevibacillus expression vector pNCMO2. The plasmids were prepared using Escherichia coli JM109.

Brevibacillus choshinensis SP3 (Takara Bio, Inc.) was transformed with each of the obtained plasmids, and the recombinant cells capable of secreting each engineered C-G29A.2d were grown. These recombinant cells were cultured with shaking for three days at 30° C. in 30 mL of A medium (3.0% polypeptone, 0.5% yeast extract, 3% glucose, 0.01% magnesium sulfate, 0.001% iron sulfate, 0.001% manganese chloride, 0.0001% zinc chloride) containing 60 μg/mL of neomycin.

The amino acid sequences of C-Q9A/G29A.2d, C-Q9S/G29A.2d, C-Q9T/G29A.2d, C-G29A/Q32A.2d, C-G29A/K35S.2d, and C-G29A/Q32T.2d expressed as above are shown in SEQ ID Nos: 10 to 15, respectively, in the Sequence Listing.

After the culture, the cells were removed from the culture medium by centrifugation (15,000 rpm at 25° C. for 5 min). Subsequently, the concentration of each engineered C-G29A.2d in the culture supernatant was measured by high performance liquid chromatography. An elution test was performed on each engineered C-G29A.2d or C-G29A.2d in the culture supernatant using an IgG-immobilized carrier under the following conditions.

<Conditions for Elution Test using IgG-Immobilized Carrier>
Carrier: IgG Sehparose FF (GE Healthcare)
Column: Omnifit column (Diba Industries); column diameter: 0.66 cm; bed height: 6.4 cm; column volume: 2.19 mL
Flow rate: 0.8 mL/min; contact time: 2.7 min
Loading volume: 470 μL (ligand concentration: 1.3 mg/mL)
Equilibration buffer: 50 mM Tris-HCl, 150 mM NaCl buffer, pH 7.5
Elution conditions: 50 mM citrate buffer (pH 6.0), followed by 50 mM citrate buffer (pH 3.0) (20 CV)

The difference between the elution pHs of C-G29A.2d (taken as reference) and each engineered C-G29A.2d was calculated. Table 1 shows the results. Each engineered C-G29A.2d eluted at a higher pH than C-G29A.2d from the IgG-immobilized carrier. These results suggest that carriers on which such engineered C-G29A.2d is immobilized can elute antibodies at higher pH than carriers with immobilized C-G29A.2d.

TABLE 1

| | Difference in elution pH |
|---|---|
| Ligand | (C-G29A.2d as reference) |
| C-Q9A/G29A.2d | 0.26 |
| C-Q9S/G29A.2d | 0.39 |
| C-Q9T/G29A.2d | 0.48 |
| C-G29A/Q32A.2d | 0.19 |
| C-G29A/Q32T.2d | 0.08 |
| C-G29A/K35S.2d | 0.06 |

### Example 2

### Evaluation of Antibody-Binding Capacity of C Domain Variant using Intermolecular Interaction Analyzer

The affinity of the various proteins obtained in Example 1 for immunoglobulin was analyzed using a surface plasmon resonance based biosensor “Biacore 3000” (GE Healthcare). In this example, a human immunoglobulin G preparation (hereinafter referred to as human IgG) fractionated from human plasma was used.

The human IgG was immobilized on a sensor chip, and each protein was flowed on the chip to detect an interaction between them. The immobilization of human IgG on the sensor chip CM5 was carried out by amine coupling using N-hydroxysuccinimide (NHS) and N-ethyl-N′-(3-dimethylaminopropyl)carbodiimide hydrochroride (EDC), and ethanolamine was used for blocking (the sensor chip and the immobilization reagents are all available from GE Healthcare). A human IgG solution was prepared by dissolving gammaglobulin “Nichiyaku” (Nihon Pharmaceutical Co. Ltd.) in a standard buffer (20 mM NaH₂PO₄—Na₂HPO₄, 150 mM NaCl, pH 7.4) to a concentration of 1.0 mg/mL. The human IgG solution was diluted by a factor of 100 in an immobilization buffer (10 mM CH₃COOH—CH₃COONa, pH 5.0), and the human IgG was immobilized onto the sensor chip in accordance with the protocol attached to the Biacore 3000. A reference cell as a negative control was also prepared by immobilizing ethanolamine onto another flow cell on the chip after activation with EDC/NHS.

Each protein was appropriately prepared at concentrations of 10 to 1,000 nM using running buffer (20 mM NaH₂PO₄—Na₂HPO₄, 150 mM NaCl, 0.005% P-20, pH 7.4) (three solutions with different protein concentrations were prepared for each protein), and each protein solution was added to the sensor chip at a flow rate of 20 μL/min for 30 seconds. Binding sensorgrams were sequentially measured at 25° C. during the addition (association phase, 30 seconds) and after the addition (dissociation phase, 60 seconds). After each measurement, the sensor chip was regenerated for 30 seconds by adding 10 mM glycine-HCl (pH 3.0, GE Healthcare). This process was intended to remove the added proteins remaining on the sensor chip, and it was confirmed that the binding activity of the immobilized human IgG was substantially completely recovered.

The binding sensorgrams (from which the binding sensorgram of the reference cell was subtracted) were subjected to fitting using the 1:1 binding model in software BIA evaluation attached to the system to calculate the association rate constant (kₒₙ), dissociation rate constant (k_{off}), and association constant (K_{A}=kₒₙ/k_{off}). Table 2 shows the results.

As shown in Table 2, the binding parameters of each engineered C-G29A.2d to human IgG were comparable to those of C-G29A.2d (control). Specifically, each ligand had an association constant with human IgG of 10⁸ M⁻¹ or more. Each engineered C-G29A.2d exhibited an antibody-binding capacity comparable to that of non-mutated C-G29A.2d in a neutral pH range.

TABLE 2

| Ligand | Kₒₙ (×10⁵ M⁻¹s) | K_{off} (×10⁻³ s⁻¹) | K_{A} (×10⁸ M⁻¹) |
|---|---|---|---|
| C-G29A.2d (control) | 2.8 | 0.9 | 3.3 |
| C-Q9A/G29A.2d | 6.1 | 1.8 | 3.3 |
| C-Q9S/G29A.2d | 6.3 | 2.0 | 3.2 |
| C-Q9T/G29A.2d | 5.5 | 2.1 | 2.6 |
| C-G29A/Q32A.2d | 4.3 | 1.1 | 3.9 |
| C-G29A/K35S.2d | 3.4 | 1.5 | 2.2 |

### Example 3

### Evaluation of Antibody-Binding Capacity of B Domain Variant using IgG-Immobilized Carrier

The total synthesis of an artificially synthesized gene of the variant B-Q9A/G29A.2d was outsourced to Eurofins Genomics K.K. The gene was synthesized by introducing a substitution of Ala for Gln at position 9 into a DNA (SEQ ID NO: 9) obtained by adding PstI and XbaI recognition sites to the 5′ and 3′ ends, respectively, of a DNA encoding B-G29A.2d (SEQ ID NO: 8) containing G29A mutation in the B domain of Protein A. Similarly to Example 1, the gene was recombinantly expressed, and the resulting culture supernatant was subjected to an elution test using an IgG-immobilized carrier. As a result, B-Q9A/G29A.2d eluted at a pH higher by 0.22 than that of B-G29A.2d from the IgG-immobilized carrier. These results suggest that the mutations indicated in Example 1 provide similar effects on the B domain, as well as on the C domain.

### Example 4

An elution test was performed on the culture supernatant of each engineered C-G29A.2d or the control C-G29A.2d obtained in Example 1 using an IgG-immobilized carrier under the following conditions.

<Conditions for Elution Test using IgG-Immobilized Carrier>
Carrier: IgG Sehparose FF (GE Healthcare)
Column: Omnifit column (Diba Industries); column diameter: 0.66 cm; bed height: 6.4 cm
Column volume: 2.19 mL
Flow rate: 0.8 mL/min; contact time: 2.7 min
Loading volume: 470 μL (ligand concentration: 1.3 mg/mL)
Equilibration buffer: 50 mM Tris-HCl, 150 mM NaCl buffer, pH 7.5
Elution conditions: Elution (1) with 50 mM citrate buffer, pH 4.0 (2 CV); Elution (2) with 50 mM citrate buffer, pH 3.0 (4 CV)

The ligand concentration of the eluates was measured to calculate the percent recovery. The results are shown in Table 3. Each engineered C-G29A.2d exhibited a higher percent recovery with an eluent having a pH of 4.0 than C-G29A.2d. It is expected from these results that carriers on which such engineered C-G29A.2d is immobilized will exhibit an increased antibody recovery when the antibody is eluted at a higher pH as compared to carriers with immobilized C-G29A.2d.

TABLE 3

| | Percent recovery (%) | |
|---|---|---|
| Ligand | Elution (1) (pH 4.0) | Elution (2) (pH 3.0) |
| C-G29A.2d (control) | 25 | 59 |
| C-Q9A/G29A.2d | 95 | 2 |
| C-Q9S/G29A.2d | 102 | 4 |
| C-Q9T/G29A.2d | 88 | 3 |
| C-G29A/Q32A.2d | 76 | 29 |
| C-G29A/K35S.2d | 37 | 49 |

### Example 5

### Antibody Elution Test using Engineered C-G29A.2d Affinity Separation Matrix

The culture of the engineered C-G29A.2d or the control C-G29A.2d obtained as in Example 1 was centrifuged to separate the cells, and acetic acid was added to the culture supernatant to adjust the pH to 4.5, followed by standing for one hour to precipitate the target protein. The precipitate was recovered by centrifugation and dissolved in a buffer (50 mM Tris-HCl, pH 8.5).

Next, the target protein was purified by anion exchange chromatography using HiTrap Q column (GE Healthcare Bio-Sciences). Specifically, the target protein solution was added to the HiTrap Q column equilibrated with an anion exchange buffer A (50 mM Tris-HCl, pH 8.0), and washed with the anion exchange buffer A, followed by elution with a salt gradient using the anion exchange buffer A and an anion exchange buffer B (50 mM Tris-HCl, 1 M NaCl, pH 8.0) to separate the target protein eluted in the middle of the gradient. The separated target protein solution was dialyzed with ultrapure water. The dialyzed aqueous solution was used as a finally purified sample. All processes of protein purification by column chromatography were carried out using AKTA avant system (GE Healthcare Bio-Sciences).

The water-insoluble base material used was a commercially available activated prepacked column “Hitrap NHS activated HP” (1 mL) (GE Healthcare). This column is a cross-linked agarose-based column into which N-hydroxysuccinimide (NHS) groups for immobilizing proteinic ligands have been introduced. Each of the finally purified samples was immobilized as a ligand to prepare affinity separation matrices in accordance with the product manual.

Specifically, the finally purified sample was diluted to a final concentration of about 13 mg/mL in a coupling buffer (0.2 M sodium carbonate, 0.5 M NaCl, pH 8.3) to prepare a solution (1 mL). Then, 2 mL of 1 mM HCl cooled in an ice bath was flowed at a flow rate of 1 mL/min. This procedure was repeated three times to remove isopropanol from the column. Immediately thereafter, 1 mL of the sample dilution solution prepared as above was added at the same flow rate. The top and bottom of the column were sealed, and the column was left at 25° C. for 30 minutes to immobilize the protein onto the column. Thereafter, the column was opened, and 3 mL of the coupling buffer was flowed at the same flow rate to recover unreacted proteins. Subsequently, 2 mL of a blocking buffer (0.5 M ethanolamine, 0.5 M NaCl, pH 8.3) was flowed. This procedure was repeated three times. Then, 2 mL of a washing buffer (0.1 M acetic acid, 0.5 M NaCl, pH 4.0) was flowed. This procedure was repeated three times. Finally, 2 mL of a standard buffer (20 mM NaH₂PO₄—Na₂HPO₄, 150 mM NaCl, pH 7.4) was flowed. Thus, the preparation of an affinity separation column was completed. An antibody elution test was performed using the affinity separation matrix under the conditions indicated below. The test was also performed using a C-G29A.2d affinity separation matrix prepared as a control in the same manner. The percentage of antibody recovery was calculated by measuring the absorbance of the eluate.

<Conditions for Antibody Elution Test using Engineered C-G29A.2d Affinity Separation Matrix>
Column: prepacked column “Hitrap NHS activated HP”, 1 mL (GE Healthcare) (column with each ligand immobilized on carrier)
Flow rate: 0.33 mL/min; contact time: 3.0 min
Loading liquid: gammaglobulin “Nichiyaku” (Nihon Pharmaceutical Co. Ltd.), 5 mL (ligand concentration: 1 mg/mL)
Equilibration buffer: Dulbecco's phosphate buffered saline (Sigma Aldrich)
Elution conditions: Elution (1) with 50 mM citrate buffer (4 CV), pH 4.0 for Test A, pH 3.75 for Test B, pH 3.5 for Test C; Elution (2) with 50 mM citrate buffer, pH 3.0 (4 CV)

The results are shown in Table 4. The affinity separation matrix prepared with C-Q9T/G29A.2d exhibited higher antibody recoveries in the eluents having a high pH (pH 4.0 to 3.5) than the affinity separation matrix with C-G29A.2d. These results suggest that the ligands that had a high percent recovery at a pH of 4.0 in the IgG Sepharose test in Example 4 can improve antibody recovery when the antibody is eluted at a high pH using an affinity separation matrix in which each of the ligands is immobilized on a water-insoluble carrier.

TABLE 4

| | | Antibody recovery (%) | |
|---|---|---|---|
| | Elution pH | C-G29A.2d (control) | C-Q9T/G29A.2d |
| Test A | Elution 1 (pH 4.0) | 54 | 92 |
| | Elution 2 (pH 3.0) | 46 | 8 |
| Test B | Elution 1 (pH 3.75) | 92 | 97 |
| | Elution 2 (pH 3.0) | 8 | 3 |
| Test C | Elution 1 (pH 3.5) | 99 | 100 |
| | Elution 2 (pH 3.0) | 1 | 0 |

Although the disclosure has been described with respect to only a limited number of embodiments, those skilled in the art, having benefit of this disclosure, will appreciate that various other embodiments may be devised without departing from the scope of the present invention. Accordingly, the scope of the present invention should be limited only by the attached claims.

What is claimed is:

1. A method for purifying an antibody-like protein, the method comprising:
adsorbing an antibody-like protein onto an affinity separation matrix by bringing the antibody-like protein into contact with the affinity separation matrix; and
eluting the antibody-like protein by bringing an eluent having a pH of 3.5 or higher into contact with the affinity separation matrix,
wherein the affinity separation matrix comprises a carrier and a ligand immobilized on the carrier,
wherein the ligand comprises an amino acid sequence derived from a sequence selected from the group consisting of SEQ ID Nos: 1 to 5,
wherein Gln or Lys in an Fc-binding site of the amino acid sequence is substituted by Ala, Ser, or Thr, and
wherein the ligand has a lower antibody-binding capacity in an acidic pH range, as compared to a ligand comprising the amino acid sequence without the substitution.

2. The purification method according to claim 1,
wherein the carrier is a water-insoluble base material.

3. The purification method according to claim 2,
wherein the water-insoluble base material is a synthetic polymer or a polysaccharide.

4. The purification method according to claim 3,
wherein the water-insoluble base material is the polysaccharide, the polysaccharide being cellulose or agarose.

5. The purification method according to claim 1,
wherein the eluent is an acidic buffer comprising at least one anion species selected from the group consisting of an acetate ion, a citrate ion, glycine, a succinate ion, a phosphate ion, and a formate ion.

6. The purification method according to claim 1,
wherein an eluate comprises a reduced amount of host cell proteins or a reduced amount of aggregates of the antibody-like protein.

7. The purification method according to claim 6,
wherein the elution of the antibody-like protein is carried out by pH gradient elution.

8. The purification method according to claim 7,
wherein the pH gradient elution is carried out with an eluent having a pH of 4 to 6.

9. The purification method according to claim 1,
wherein the antibody-like protein is a mixture comprising host cell proteins.

10. The purification method according to claim 1,
wherein the antibody-like protein is a mixture comprising aggregates of the antibody-like protein.


SEQUENCE LISTING (15 sequences)

SEQ ID NO: 1 - protein, 56 aa, Staphylococcus aureus
AQHDEAQQNAFYQVLNMPNLNADQRNGFIQSLKDDPSQSANVLGEAQKLNDSQAPK

SEQ ID NO: 2 - protein, 61 aa, Staphylococcus aureus
ADAQQNKFNKDQQSAFYEILNMPNLNEEQRNGFIQSLKDDPSQSTNVLGEAKKLNESQAPK

SEQ ID NO: 3 - protein, 58 aa, Staphylococcus aureus
ADNNFNKEQQNAFYEILNMPNLNEEQRNGFIQSLKDDPSQSANLLAEAKKLNESQAPK

SEQ ID NO: 4 - protein, 58 aa, Staphylococcus aureus [sequence omitted]

SEQ ID NO: 5 - protein, 58 aa, Staphylococcus aureus
ADNKFNKEQQNAFYEILHLPNLTEEQRNGFIQSLKDDPSVSKEILAEAKKLNDAQAPK

SEQ ID NO: 6 - protein, 116 aa, Artificial Sequence
  Synthetic peptide derived from C domain of Protein A of Staphylococcus aureus
ADNKFNKEQQNAFYEILHLPNLTEEQRNAFIQSLKDDPSVSKEILAEAKKLNDAQAPKADNKFNKEQQNAFYEILHLPNLTEEQRNAFIQSLKDDPSVSKEILAEAKKLNDAQAPK

SEQ ID NO: 7 - DNA, 359 bp, Artificial
  Synthetic DNA derived from C domain of Protein A of Staphylococcus aureus
ctgcagataacaaatttaacaaagaacaacaaaacgctttctacgaaatcctgcacttgccaaaccttactgaagaacaacgtaatgctttcatccaatccctgaaagatgatccatctgtatccaaagaaattttggcagaggctaaaaaacttaacgacgctcaggcgcctaaggctgataacaaattcaataaagaacagcaaaacgctttttatgaaatccttcacctgccaaatcttacagaagaacaacgcaacgcattcattcaaagcttgaaggatgacccttccgttagcaaagagatcctggctgaagcaaaaaagttgaatgatgcgcaagcaccaaaataatctaga

SEQ ID NO: 8 - protein, 116 aa, Artificial
  Synthetic peptide derived from B domain of Protein A of Staphylococcus aureus
ADNKFNKEQQNAFYEILHLPNLNEEQRNAFIQSLKDDPSQSANLLAEAKKLNDAQAPKADNKFNKEQQNAFYEILHLPNLNEEQRNAFIQSLKDDPSQSANLLAEAKKLNDAQAPK

SEQ ID NO: 9 - DNA, 359 bp, Artificial
  Synthetic DNA derived from B domain of Protein A of Staphylococcus aureus
ctgcagataacaaatttaacaaagaacaacaaaacgctttctacgaaatcctgcacttgccaaaccttaatgaagaacaacgtaatgctttcatccaatccctgaaagatgatccatctcaatccgctaaccttttggcagaggctaaaaaacttaacgacgctcaggcgcctaaggctgataacaaattcaataaagaacagcaaaacgctttttatgaaatccttcacctgccaaatcttaacgaagaacaacgcaacgcattcattcaaagcttgaaggatgacccttcccaaagcgcaaatttgctggctgaagcaaaaaagttgaatgatgcgcaagcaccaaaataatctaga

SEQ ID NO: 10 - protein, 116 aa, Artificial Sequence
  Synthetic peptide derived from C domain of Protein A of Staphylococcus aureus (C-Q9A/G29A.2d)
ADNKFNKEAQNAFYEILHLPNLTEEQRNAFIQSLKDDPSVSKEILAEAKKLNDAQAPKADNKFNKEAQNAFYEILHLPNLTEEQRNAFIQSLKDDPSVSKEILAEAKKLNDAQAPK

SEQ ID NO: 11 - protein, 116 aa, Artificial Sequence
  Synthetic peptide derived from C domain of Protein A of Staphylococcus aureus (C-Q9S/G29A.2d)
ADNKFNKESQNAFYEILHLPNLTEEQRNAFIQSLKDDPSVSKEILAEAKKLNDAQAPKADNKFNKESQNAFYEILHLPNLTEEQRNAFIQSLKDDPSVSKEILAEAKKLNDAQAPK

SEQ ID NO: 12 - protein, 116 aa, Artificial Sequence
  Synthetic peptide derived from C domain of Protein A of Staphylococcus aureus (C-Q9T/G29A.2d)
ADNKFNKETQNAFYEILHLPNLTEEQRNAFIQSLKDDPSVSKEILAEAKKLNDAQAPKADNKFNKETQNAFYEILHLPNLTEEQRNAFIQSLKDDPSVSKEILAEAKKLNDAQAPK

SEQ ID NO: 13 - protein, 116 aa, Artificial Sequence
  Synthetic peptide derived from C domain of Protein A of Staphylococcus aureus (C-G29A/Q32A.2d)
ADNKFNKEQQNAFYEILHLPNLTEEQRNAFIASLKDDPSVSKEILAEAKKLNDAQAPKADNKFNKEQQNAFYEILHLPNLTEEQRNAFIASLKDDPSVSKEILAEAKKLNDAQAPK

SEQ ID NO: 14 - protein, 116 aa, Artificial Sequence
  Synthetic peptide derived from C domain of Protein A of Staphylococcus aureus (C-G29A/K35S.2d)
ADNKFNKEQQNAFYEILHLPNLTEEQRNAFIQSLSDDPSVSKEILAEAKKLNDAQAPKADNKFNKEQQNAFYEILHLPNLTEEQRNAFIQSLSDDPSVSKEILAEAKKLNDAQAPK

SEQ ID NO: 15 - protein, 116 aa, Artificial Sequence
  Synthetic peptide derived from C domain of Protein A of Staphylococcus aureus (C-G29A/Q32T.2d)
ADNKFNKEQQNAFYEILHLPNLTEEQRNAFITSLKDDPSVSKEILAEAKKLNDAQAPKADNKFNKEQQNAFYEILHLPNLTEEQRNAFITSLKDDPSVSKEILAEAKKLNDAQAPK